(19) United States
(12) Patent Application Publication
(10) Pub. No.: US 2021/0338694 A1
(43) Pub. Date: Nov. 4, 2021

(54) SOS1 ALLOSTERIC SITE TARGETING

(71) Applicants: Yi Zheng, Cincinnati, OH (US); Chris Evelyn, Cincinnati, OH (US); William Seibel, Liberty Township, OH (US); Jaroslaw Meller, Cincinnati, OH (US); Jing Zhang, Madison, WI (US)

(72) Inventors: Yi Zheng, Cincinnati, OH (US); Chris Evelyn, Cincinnati, OH (US); William Seibel, Liberty Township, OH (US); Jaroslaw Meller, Cincinnati, OH (US); Jing Zhang, Madison, WI (US)

(21) Appl. No.: 17/306,646

(22) Filed: May 3, 2021

Related U.S. Application Data
(60) Provisional application No. 63/019,746, filed on May 4, 2020.

(51) Int. Cl. A61K 31/609; A61K 31/04; A61K 31/341; A61K 31/12; A61K 31/166; A61K 31/167; A61K 31/423; A61K 31/428; A61K 31/085; A61K 31/195; A61K 31/196; A61P 35/00

(52) U.S. Cl. CPC A61K 31/609; A61K 31/04; A61K 31/341; A61K 31/12; A61K 31/166; A61P 35/00; A61K 31/423; A61K 31/428; A61K 31/085; A61K 31/195; A61K 31/196; A61K 31/167

(57) ABSTRACT

Disclosed herein are small molecule SOS1 allosteric site inhibitors, pharmaceutical compositions, and methods of use thereof.

### INCORPORATION BY REFERENCE TO ANY PRIORITY APPLICATIONS

This application claims the benefit of U.S. Provisional Application No. 63/019,746, filed on May 4, 2020.

### BACKGROUND

### Field

The present invention relates to the fields of chemistry and medicine. More specifically, the present invention relates to compounds and compositions useful as inhibitors of Ras signaling in cells and their methods of use.

### Description of the Related Technology

Ras GTPases regulate intracellular signaling involved in cell proliferation. H-, N-, and K-Ras are the founding members of the Ras superfamily of small-GTPases, and are known to transduce signals from various cellular receptors including receptor tyrosine kinases, G-protein coupled receptors, and cytokine receptors, to engage in multiple intracellular signaling pathways leading to cell proliferation, differentiation, survival, and gene expression. Gain of function mutations of H-, N-, K-Ras and components of Ras signaling (e.g., EGFR, BCR-Abl, MEK, PI3K, and AKT) have been identified in human cancers. In addition, aberrant Ras signaling is involved in several developmental disorders, known as the Cardio-Facio-Cutaneous (CFC) diseases (i.e. NF-1, Costello syndrome, and Noonan syndrome). Ras activity in cells is regulated by two classes of enzymes that control its guanine nucleotide bound states—the activators, guanine nucleotide exchange factors (GEFs), that stimulate the exchange of Ras bound GDP for GTP and enable Ras to interact with their effectors leading to downstream signaling, and the inactivators, GTPase-activating proteins (GAPs), that stimulate GTP-hydrolysis and switch off Ras signaling.

There are three subfamilies of Ras GEFs—SOS, Ras-GRF, and Ras-GRP, that are expressed in different cell-types. In particular, SOS1 is a ubiquitously expressed multi-domain enzyme with a REM domain and a Cdc25 catalytic domain essential for Ras GTPase activation and signaling. The REM and Cdc25 domains contain two distinct sites for Ras binding—a catalytic site and an allosteric site. The catalytic site binds GDP-bound Ras and stimulates GDP/GTP exchange on Ras. An increase in SOS1 mutations has been identified in patients with the developmental disorder Noonan syndrome and genetic disorder hereditary gingival fibromatosis type 1, and SOS1 has been shown to be differentially expressed in prostate and breast cancer samples. In addition, SOS1 has been recognized as a critical nodal mediating signal flow from receptor tyrosine kinases to Ras and downstream ERK and PI3K cascades.

Strategies targeting Ras signaling downstream components have yielded compounds that bear considerable toxicity. Direct inhibition of Ras mutants has proved extremely challenging. Based on the failure of all direct and indirect approaches so far, oncogenic Ras is generally considered undruggable.

### SUMMARY

Some embodiments described herein relate to a pharmaceutical composition comprising a compound of Formula I:

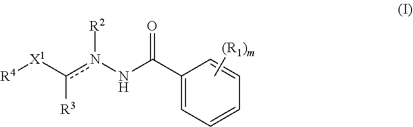

wherein each R¹ and R⁴ is independently selected from the group consisting of halogen, an optionally substituted alkyl, an optionally substituted alkenyl, an optionally substituted alkynyl, an optionally substituted aryl, an optionally substituted heteroaryl, —COOH, alkoxy, —OH, and —NO₂; R² is hydrogen or absent; “--” is a pi bond or absent; R³ is hydrogen, ═O, ═S, an optionally substituted aryl, or an optionally substituted heteroaryl; m is 0, 1, 2, or 3; and X¹ is a covalent bond,

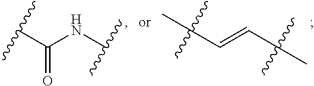

or a stereoisomer or pharmaceutically acceptable salt thereof, and a pharmaceutically acceptable excipient.

In some embodiments, each R¹ is independently selected from the group consisting of —OH, Br, F, and phenyl. In some embodiments, R¹ is Br. In some embodiments, R¹ is F. In some embodiments, m is 1. In some embodiments, m is 2.

Additional embodiments relate to a pharmaceutical composition comprising a compound having the structure of Formula Ia:

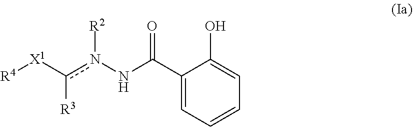

or a stereoisomer or pharmaceutically acceptable salt thereof, and a pharmaceutically acceptable excipient. In some embodiments, “--” is a pi bond and R² is absent. In some embodiments, R² is hydrogen and “--” is absent. In some embodiments, R³ is hydrogen. In some embodiments, R³ is phenyl. In some embodiments, R³ is ═O. In some embodiments, R³ is ═S.

Some embodiments described herein relate to a pharmaceutical composition comprising a compound having the structure of Formula Ib:

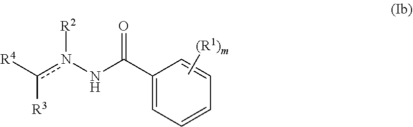

or a stereoisomer or pharmaceutically acceptable salt thereof. In some embodiments, X¹ is

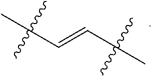 [SMILES: */C=C/*]

In some embodiments, R⁴ is optionally substituted naphthyl. In some embodiments, R⁴ is optionally substituted furanyl.

Additional embodiments described herein comprise a compound having the structure of Formula Ic:

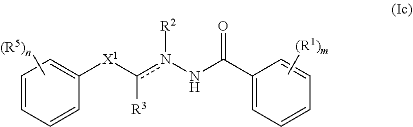

wherein each R⁵ is independently selected from the group consisting of halogen, —COOH, —OH, —NO₂, phenyl,

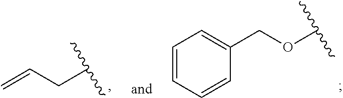

and n is 0, 1, 2, or 3;

or a stereoisomer or pharmaceutically acceptable salt thereof. In some embodiments, R⁵ is independently selected from the group consisting of Br, —COOH, —OH, and —NO₂. In some embodiments, n is 0. In some embodiments, n is 1. In some embodiments, n is 2.

In further embodiments, the compound is selected from:

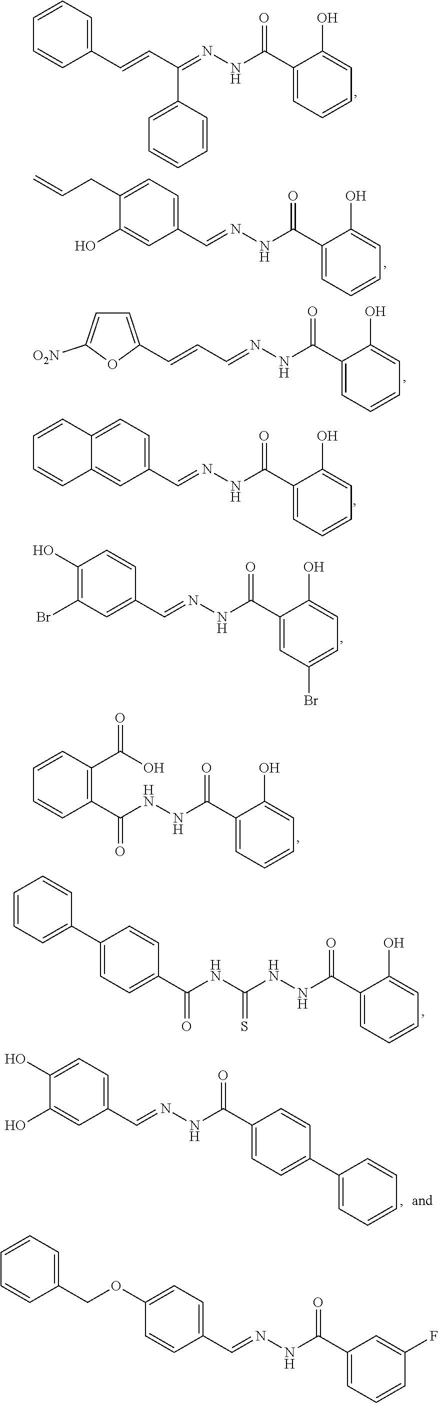

or a stereoisomer or pharmaceutically acceptable salt thereof.

Additional embodiments described herein relate to a pharmaceutical composition comprising a compound selected from:

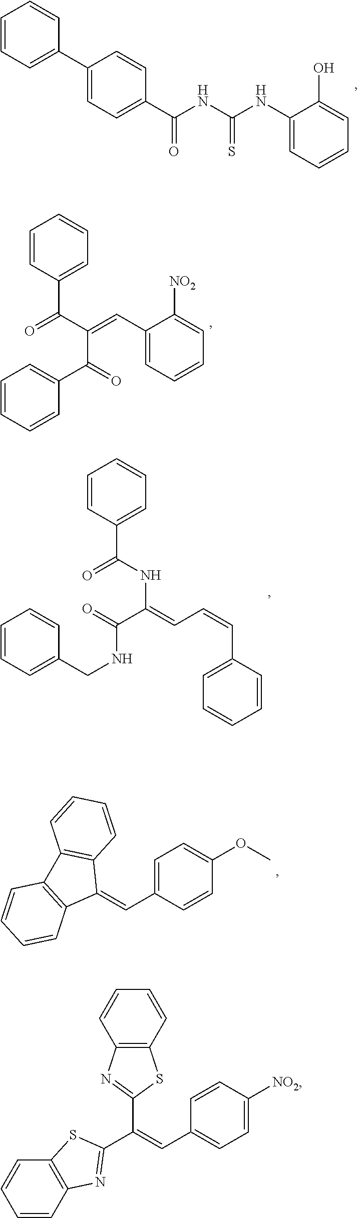 [SMILES: O=C(NC(=S)Nc1ccccc1O)c1ccc(-c2ccccc2)cc1 ; O=C(C(=Cc1ccccc1[N+](=O)[O-])C(=O)c1ccccc1)c1ccccc1 ; O=C(NCc1ccccc1)/C(=C/C=C\c1ccccc1)NC(=O)c1ccccc1 ; COc1ccc(C=C2c3ccccc3-c3ccccc32)cc1 ; O=[N+]([O-])c1ccc(C=C(c2nc3ccccc3s2)c2nc3ccccc3s2)cc1]

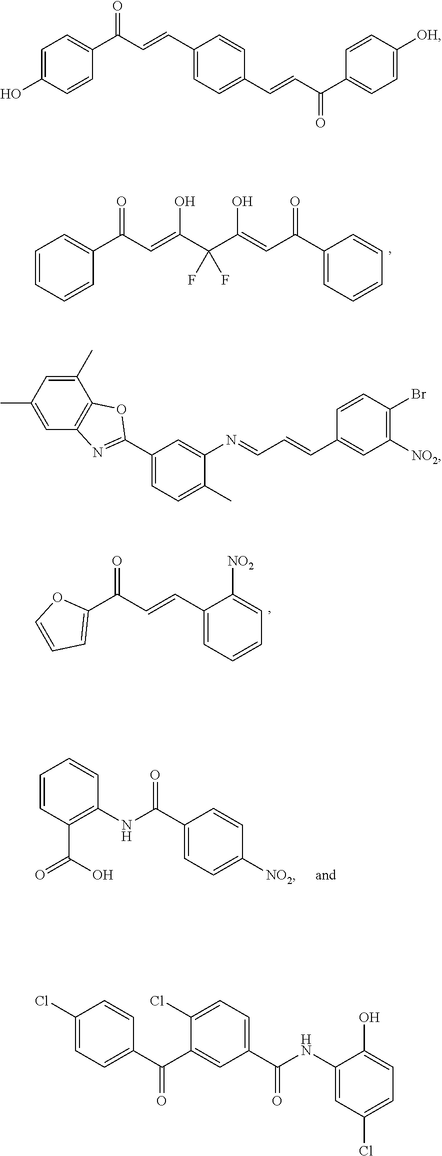 [SMILES: O=C(/C=C/c1ccc(/C=C/C(=O)c2ccc(O)cc2)cc1)c1ccc(O)cc1 ; O=C(/C=C(\O)C(F)(F)/C(O)=C/C(=O)c1ccccc1)c1ccccc1 ; Cc1cc(C)c2oc(-c3ccc(C)c(/N=C/C=C/c4ccc(Br)c([N+](=O)[O-])c4)c3)nc2c1 ; O=C(/C=C/c1ccccc1[N+](=O)[O-])c1ccco1 ; O=C(Nc1ccccc1C(=O)O)c1ccc([N+](=O)[O-])cc1 ; O=C(Nc1cc(Cl)ccc1O)c1ccc(Cl)c(C(=O)c2ccc(Cl)cc2)c1]

or a stereoisomer or pharmaceutically acceptable salt thereof, and a pharmaceutically acceptable excipient.

Further embodiments relate to a pharmaceutical composition comprising a therapeutically effective amount of a compound as described herein and a pharmaceutically acceptable excipient. In some embodiments, the therapeutically effective amount is an amount effective for allosteric inhibition of SOS1. In some embodiments, the therapeutically effective amount is an amount effective for allosteric inhibition of SOS1 in an oncogenic cell. In some embodiments, the oncogenic cell is present due to a Ras-mediated cancer. In some embodiments, the Ras-mediated cancer is pancreatic cancer or acute myeloid leukemia.

Additional embodiments relate to a method of treating cancer, comprising administering to a subject in need thereof a compound as described herein.

Further embodiments relate to a method of testing a compound for an ability to inhibit a Ras-mediated cancer comprising: contacting a Ras-mediated cancer cell culture with a test compound; determining the percent cell survival of the cells within the Ras-mediated cancer cell culture; identifying a compound as having an ability to inhibit a Ras-mediated cancer when the percent cell survival of the cells within the Ras-mediated cancer cell culture is below a threshold amount.

### BRIEF DESCRIPTION OF THE DRAWINGS

FIG. 1 is a graph showing the results of an experiment in which pancreatic cancer cells were exposed to NSC-70220.

FIG. 2A is a Western blot showing the results of an experiment in which pancreatic cancer cells were exposed to NSC-70220.

FIG. 2B is a Western blot showing the results of an experiment in which pancreatic cancer cells were exposed to NSC-70220.

FIG. 3 is a graph showing the results of an experiment in which pancreatic cancer cells were exposed to NSC-70220.

FIG. 4 is a graph showing the results of an experiment in which pancreatic cancer cells were exposed to NSC-70220.

### DETAILED DESCRIPTION

SOS1 and SOS2 are the major Ras GEFs ubiquitously expressed in many cell types including hematopoietic cells. They share an almost identical catalytic domain (Soscat) that contains a REM domain and a Cdc25 homology domain. SOS1 and SOS2 have two distinct binding sites on Soscat: the allosteric site binds to active Ras (Ras-GTP), while the catalytic site binds to Ras-GDP. The present inventors have postulated that small molecule targeting of the allosteric site of SOS1 may be useful for selectively suppressing oncogenic Kras activity.

### Definitions

Unless defined otherwise, all technical and scientific terms used herein have the same meaning as is commonly understood by one of ordinary skill in the art to which this disclosure belongs. All patents, applications, published applications, and other publications are incorporated by reference in their entirety. In the event that there is a plurality of definitions for a term herein, those in this section prevail unless stated otherwise.

The term “pharmaceutically acceptable salt” refers to salts that retain the biological effectiveness and properties of a compound, which are not biologically or otherwise undesirable for use in a pharmaceutical. In many cases, the compounds herein are capable of forming acid and/or base salts by virtue of the presence of amino and/or carboxyl groups or groups similar thereto. Pharmaceutically acceptable acid addition salts can be formed with inorganic acids and organic acids. Inorganic acids from which salts can be derived include, for example, hydrochloric acid, hydrobromic acid, sulfuric acid, nitric acid, phosphoric acid, and the like. Organic acids from which salts can be derived include, for example, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like. Pharmaceutically acceptable base addition salts can be formed with inorganic and organic bases. Inorganic bases from which salts can be derived include, for example, sodium, potassium, lithium, ammonium, calcium, magnesium, iron, zinc, copper, manganese, aluminum, and the like; particularly preferred are the ammonium, potassium, sodium, calcium and magnesium salts. Organic bases from which salts can be derived include, for example, primary, secondary, and tertiary amines, substituted amines including naturally occurring substituted amines, cyclic amines, basic ion exchange resins, and the like, specifically such as isopropylamine, trimethylamine, diethylamine, triethylamine, tripropylamine, and ethanolamine. Many such salts are known in the art, as described in WO 87/05297, Johnston et al., published Sep. 11, 1987 (incorporated by reference herein in its entirety).

As used herein, “Cₐ to C_{b}” or “C_{a-b}” in which “a” and “b” are integers refer to the number of carbon atoms in the specified group. That is, the group can contain from “a” to “b”, inclusive, carbon atoms. Thus, for example, a “C₁ to C₄ alkyl” or “C₁₋₄ alkyl” group refers to all alkyl groups having from 1 to 4 carbons, that is, CH₃—, CH₃CH₂—, CH₃CH₂CH₂—, (CH₃)₂CH—, CH₃CH₂CH₂CH₂—, CH₃CH₂CH(CH₃)— and (CH₃)₃C—.

The term “halogen” or “halo,” as used herein, means any one of the radio-stable atoms of column 7 of the Periodic Table of the Elements, e.g., fluorine, chlorine, bromine, or iodine, with fluorine and chlorine being preferred.

As used herein, “alkyl” refers to a straight or branched hydrocarbon chain that is fully saturated (i.e., contains no double or triple bonds). The alkyl group may have 1 to 20 carbon atoms (whenever it appears herein, a numerical range such as “1 to 20” refers to each integer in the given range; e.g., “1 to 20 carbon atoms” means that the alkyl group may consist of 1 carbon atom, 2 carbon atoms, 3 carbon atoms, etc., up to and including 20 carbon atoms, although the present definition also covers the occurrence of the term “alkyl” where no numerical range is designated). The alkyl group may also be a medium size alkyl having 1 to 9 carbon atoms. The alkyl group could also be a lower alkyl having 1 to 4 carbon atoms. The alkyl group of the compounds may be designated as “C₁₋₄ alkyl” or similar designations. By way of example only, “C₁₋₄ alkyl” indicates that there are one to four carbon atoms in the alkyl chain, i.e., the alkyl chain is selected from the group consisting of methyl, ethyl, propyl, iso-propyl, n-butyl, iso-butyl, sec-butyl, and t-butyl. Typical alkyl groups include, but are in no way limited to, methyl, ethyl, propyl, isopropyl, butyl, isobutyl, tertiary butyl, pentyl, hexyl, and the like.

As used herein, “haloalkyl” refers to a straight- or branched-chain alkyl group having from 1 to 12 carbon atoms in the chain, substituting one or more hydrogens with halogens. Examples of haloalkyl groups include, but are not limited to, —CF₃, —CHF₂, —CH₂F, —CH₂CF₃, —CH₂CHF₂, —CH₂CH₂F, —CH₂CH₂C₁, —CH₂CF₂CF₃ and other groups that in light of the ordinary skill in the art and the teachings provided herein, would be considered equivalent to any one of the foregoing examples.

As used herein, “alkoxy” refers to the formula —OR wherein R is an alkyl as is defined above, such as “C₁₋₉ alkoxy”, including but not limited to methoxy, ethoxy, n-propoxy, 1-methylethoxy (isopropoxy), n-butoxy, iso-butoxy, sec-butoxy, and tert-butoxy, and the like.

As used herein, “polyethylene glycol” refers to the formula

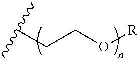

wherein n is an integer greater than one and R is a hydrogen or alkyl. The number of repeat units “n” may be indicated by referring to a number of members. Thus, for example, “2- to 5-membered polyethylene glycol” refers to n being an integer selected from two to five. In some embodiments, R is selected from methoxy, ethoxy, n-propoxy, 1-methylethoxy (isopropoxy), n-butoxy, iso-butoxy, sec-butoxy, and tert-butoxy.

As used herein, “heteroalkyl” refers to a straight or branched hydrocarbon chain containing one or more heteroatoms, that is, an element other than carbon, including but not limited to, nitrogen, oxygen and sulfur, in the chain backbone. The heteroalkyl group may have 1 to 20 carbon atoms although the present definition also covers the occurrence of the term “heteroalkyl” where no numerical range is designated. The heteroalkyl group may also be a medium size heteroalkyl having 1 to 9 carbon atoms. The heteroalkyl group could also be a lower heteroalkyl having 1 to 4 carbon atoms. In various embodiments, the heteroalkyl may have from 1 to 4 heteroatoms, from 1 to 3 heteroatoms, 1 or 2 heteroatoms, or 1 heteroatom. The heteroalkyl group of the compounds may be designated as “C₁₋₄ heteroalkyl” or similar designations. The heteroalkyl group may contain one or more heteroatoms. By way of example only, “C₁₋₄ heteroalkyl” indicates that there are one to four carbon atoms in the heteroalkyl chain and additionally one or more heteroatoms in the backbone of the chain.

The term “aromatic” refers to a ring or ring system having a conjugated pi electron system and includes both carbocyclic aromatic (e.g., phenyl) and heterocyclic aromatic groups (e.g., pyridine). The term includes monocyclic or fused-ring polycyclic (i.e., rings which share adjacent pairs of atoms) groups provided that the entire ring system is aromatic.

As used herein, “aryl” refers to an aromatic ring or ring system (i.e., two or more fused rings that share two adjacent carbon atoms) containing only carbon in the ring backbone. When the aryl is a ring system, every ring in the system is aromatic. The aryl group may have 6 to 18 carbon atoms, although the present definition also covers the occurrence of the term “aryl” where no numerical range is designated. In some embodiments, the aryl group has 6 to 10 carbon atoms. The aryl group may be designated as “C₆₋₁₀ aryl,” “C₆ or C₁₀ aryl,” or similar designations. Examples of aryl groups include, but are not limited to, phenyl, naphthyl, azulenyl, and anthracenyl.

As used herein, “aryloxy” and “arylthio” refers to RO— and RS—, in which R is an aryl as is defined above, such as “C₆₋₁₀ aryloxy” or “C₆₋₁₀ arylthio” and the like, including but not limited to phenyloxy.

An “aralkyl” or “arylalkyl” is an aryl group connected, as a substituent, via an alkylene group, such “C₇₋₁₄ aralkyl” and the like, including but not limited to benzyl, 2-phenylethyl, 3-phenylpropyl, and naphthylalkyl. In some cases, the alkylene group is a lower alkylene group (i.e., a C₁₋₄ alkylene group).

As used herein, “heteroaryl” refers to an aromatic ring or ring system (i.e., two or more fused rings that share two adjacent atoms) that contain(s) one or more heteroatoms, that is, an element other than carbon, including but not limited to, nitrogen, oxygen and sulfur, in the ring backbone. When the heteroaryl is a ring system, every ring in the system is aromatic. The heteroaryl group may have 5-18 ring members (i.e., the number of atoms making up the ring backbone, including carbon atoms and heteroatoms), although the present definition also covers the occurrence of the term “heteroaryl” where no numerical range is designated. In some embodiments, the heteroaryl group has 5 to 10 ring members or 5 to 7 ring members. The heteroaryl group may be designated as “5-7 membered heteroaryl,” “5-10 membered heteroaryl,” or similar designations. In various embodiments, a heteroaryl contains from 1 to 4 heteroatoms, from 1 to 3 heteroatoms, from 1 to 2 heteroatoms, or 1 heteroatom. For example, in various embodiments, a heteroaryl contains 1 to 4 nitrogen atoms, 1 to 3 nitrogen atoms, 1 to 2 nitrogen atoms, 2 nitrogen atoms and 1 sulfur or oxygen atom, 1 nitrogen atom and 1 sulfur or oxygen atom, or 1 sulfur or oxygen atom. Examples of heteroaryl rings include, but are not limited to, furyl, thienyl, phthalazinyl, pyrrolyl, oxazolyl, thiazolyl, imidazolyl, pyrazolyl, isoxazolyl, isothiazolyl, triazolyl, thiadiazolyl, pyridinyl, pyridazinyl, pyrimidinyl, pyrazinyl, triazinyl, quinolinyl, isoquinlinyl, benzimidazolyl, benzoxazolyl, benzothiazolyl, indolyl, isoindolyl, and benzothienyl.

A “heteroaralkyl” or “heteroarylalkyl” is heteroaryl group connected, as a substituent, via an alkylene group. Examples include but are not limited to 2-thienylmethyl, 3-thienylmethyl, furylmethyl, thienylethyl, pyrrolylalkyl, pyridylalkyl, isoxazollylalkyl, and imidazolylalkyl. In some cases, the alkylene group is a lower alkylene group (i.e., a C₁₋₄ alkylene group).

As used herein, “carbocyclyl” means a non-aromatic cyclic ring or ring system containing only carbon atoms in the ring system backbone. When the carbocyclyl is a ring system, two or more rings may be joined together in a fused, bridged or spiro-connected fashion. Carbocyclyls may have any degree of saturation provided that at least one ring in a ring system is not aromatic. Thus, carbocyclyls include cycloalkyls, cycloalkenyls, and cycloalkynyls. The carbocyclyl group may have 3 to 20 carbon atoms, although the present definition also covers the occurrence of the term “carbocyclyl” where no numerical range is designated. The carbocyclyl group may also be a medium size carbocyclyl having 3 to 10 carbon atoms. The carbocyclyl group could also be a carbocyclyl having 3 to 6 carbon atoms. The carbocyclyl group may be designated as “C₃₋₆ carbocyclyl” or similar designations. Examples of carbocyclyl rings include, but are not limited to, cyclopropyl, cyclobutyl, cyclopentyl, cyclohexyl, cyclohexenyl, 2,3-dihydro-indene, bicycle[2.2.2]octanyl, adamantyl, and spiro[4.4]nonanyl.

A “(carbocyclyl)alkyl” is a carbocyclyl group connected, as a substituent, via an alkylene group, such as “C₄₋₁₀ (carbocyclyl)alkyl” and the like, including but not limited to, cyclopropylmethyl, cyclobutylmethyl, cyclopropylethyl, cyclopropylbutyl, cyclobutylethyl, cyclopropylisopropyl, cyclopentylmethyl, cyclopentylethyl, cyclohexylmethyl, cyclohexylethyl, cycloheptylmethyl, and the like. In some cases, the alkylene group is a lower alkylene group.

As used herein, “cycloalkyl” means a fully saturated carbocyclyl ring or ring system. Examples include cyclopropyl, cyclobutyl, cyclopentyl, and cyclohexyl.

As used herein, “cycloalkenyl” means a carbocyclyl ring or ring system having at least one double bond, wherein no ring in the ring system is aromatic. An example is cyclohexenyl.

As used herein, “heterocyclyl” means a non-aromatic cyclic ring or ring system containing at least one heteroatom in the ring backbone. Heterocyclyls may be joined together in a fused, bridged or spiro-connected fashion. Heterocyclyls may have any degree of saturation provided that at least one ring in the ring system is not aromatic. The heteroatom(s) may be present in either a non-aromatic or aromatic ring in the ring system. The heterocyclyl group may have 3 to 20 ring members (i.e., the number of atoms making up the ring backbone, including carbon atoms and heteroatoms), although the present definition also covers the occurrence of the term “heterocyclyl” where no numerical range is designated. The heterocyclyl group may also be a medium size heterocyclyl having 3 to 10 ring members. The heterocyclyl group could also be a heterocyclyl having 3 to 6 ring members. The heterocyclyl group may be designated as “3-6 membered heterocyclyl” or similar designations.

In various embodiments, a heterocyclyl contains from 1 to 4 heteroatoms, from 1 to 3 heteroatoms, from 1 to 2 heteroatoms, or 1 heteroatom. For example, in various embodiments, a heterocyclyl contains 1 to 4 nitrogen atoms, 1 to 3 nitrogen atoms, 1 to 2 nitrogen atoms, 2 nitrogen atoms and 1 sulfur or oxygen atom, 1 nitrogen atom and 1 sulfur or oxygen atom, or 1 sulfur or oxygen atom. In preferred six membered monocyclic heterocyclyls, the heteroatom(s) are selected from one up to three of O, N or S, and in preferred five membered monocyclic heterocyclyls, the heteroatom(s) are selected from one or two heteroatoms selected from O, N, or S. Examples of heterocyclyl rings include, but are not limited to, azepinyl, acridinyl, carbazolyl, cinnolinyl, dioxolanyl, imidazolinyl, imidazolidinyl, morpholinyl, oxiranyl, oxepanyl, thiepanyl, piperidinyl, piperazinyl, dioxopiperazinyl, pyrrolidinyl, pyrrolidonyl, pyrrolidionyl, 4-piperidonyl, pyrazolinyl, pyrazolidinyl, 1,3-dioxinyl, 1,3-dioxanyl, 1,4-dioxinyl, 1,4-dioxanyl, 1,3-oxathianyl, 1,4-oxathiinyl, 1,4-oxathianyl, 2H-1,2-oxazinyl, trioxanyl, hexahydro-1,3,5-triazinyl, 1,3-dioxolyl, 1,3-dioxolanyl, 1,3-dithiolyl, 1,3-dithiolanyl, isoxazolinyl, isoxazolidinyl, oxazolinyl, oxazolidinyl, oxazolidinonyl, thiazolinyl, thiazolidinyl, 1,3-oxathiolanyl, indolinyl, isoindolinyl, tetrahydrofuranyl, tetrahydropyranyl, tetrahydrothiophenyl, tetrahydrothiopyranyl, tetrahydro-1,4-thiazinyl, thiamorpholinyl, dihydrobenzofuranyl, benzimidazolidinyl, and tetrahydroquinoline.

A “(heterocyclyl)alkyl” is a heterocyclyl group connected, as a substituent, via an alkylene group. Examples include, but are not limited to, imidazolinylmethyl and indolinylethyl.

As used herein, “acyl” refers to —C(═O)R, wherein R is hydrogen, C₁₋₆ alkyl, C₂₋₆ alkenyl, C₂₋₆ alkynyl, C₃₋₇ carbocyclyl, aryl, 5-10 membered heteroaryl, and 5-10 membered heterocyclyl, as defined herein. Non-limiting examples include formyl, acetyl, propanoyl, benzoyl, and acryl.

An “O-carboxy” group refers to a “—OC(═O)R” group in which R is selected from hydrogen, C₁₋₆ alkyl, C₂₋₆ alkenyl, C₂₋₆ alkynyl, C₃₋₇ carbocyclyl, aryl, 5-10 membered heteroaryl, and 5-10 membered heterocyclyl, as defined herein.

A “C-carboxy” group refers to a “—C(═O)OR” group in which R is selected from hydrogen, C₁₋₆ alkyl, C₂₋₆ alkenyl, C₂₋₆ alkynyl, C₃₋₇ carbocyclyl, aryl, 5-10 membered heteroaryl, and 5-10 membered heterocyclyl, as defined herein. A non-limiting example includes carboxyl (i.e., —C(═O)OH).

A “cyano” group refers to a “—CN” group.

A “cyanato” group refers to an “—OCN” group.

An “isocyanato” group refers to a “—NCO” group.

A “thiocyanato” group refers to a “—SCN” group.

An “isothiocyanato” group refers to an “—NCS” group.

A “sulfinyl” group refers to an “—S(═O)R” group in which R is selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 5-10 membered heterocyclyl, as defined herein.

A “sulfonyl” group refers to an “—SO₂R” group in which R is selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 5-10 membered heterocyclyl, as defined herein.

An “S-sulfonamido” group refers to a “—SO₂NR_{A}R_{B}” group in which R_{A} and R_{B} are each independently selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 5-10 membered heterocyclyl, as defined herein.

An “N-sulfonamido” group refers to a “—N(R_{A})SO₂R_{B}” group in which R_{A} and R_{b} are each independently selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 5-10 membered heterocyclyl, as defined herein.

An “O-carbamyl” group refers to a “—OC(═O)NR_{A}R_{B}” group in which R_{A} and R_{B} are each independently selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 5-10 membered heterocyclyl, as defined herein.

An “N-carbamyl” group refers to an “—N(R_{A})OC(═O)R_{B}” group in which R_{A} and R_{B} are each independently selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 5-10 membered heterocyclyl, as defined herein.

An “O-thiocarbamyl” group refers to a “—OC(═S)NR_{A}R_{B}” group in which R_{A} and R_{B} are each independently selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 5-10 membered heterocyclyl, as defined herein.

An “N-thiocarbamyl” group refers to an “—N(R_{A})OC(═S)R_{B}” group in which R_{A} and R_{B} are each independently selected from hydrogen, C₁₋₆ alkyl, C₂₋₆ alkenyl, C₂-6 alkynyl, C₃₋₇ carbocyclyl, C₆₋₁₀ aryl, 5-10 membered heteroaryl, and 5-10 membered heterocyclyl, as defined herein.

A “C-amido” group refers to a “—C(═O)NR_{A}R_{B}” group in which R_{A} and R_{B} are each independently selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 5-10 membered heterocyclyl, as defined herein.

An “N-amido” group refers to a “—N(R_{A})C(═O)R_{B}” group in which R_{A} and R_{B} are each independently selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 5-10 membered heterocyclyl, as defined herein.

An “amino” group refers to a “—NR_{A}R_{B}” group in which R_{A} and R_{B} are each independently selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 5-10 membered heterocyclyl, as defined herein.

An “aminoalkyl” group refers to an amino group connected via an alkylene group.

An “alkoxyalkyl” group refers to an alkoxy group connected via an alkylene group, such as a “C₂₋₈ alkoxyalkyl” and the like.

As used herein, a “natural amino acid side chain” refers to the side-chain substituent of a naturally occurring amino acid. Naturally occurring amino acids have a substituent attached to the α-carbon. Naturally occurring amino acids include Arginine, Lysine, Aspartic acid, Glutamic acid, Glutamine, Asparagine, Histidine, Serine, Threonine, Tyrosine, Cysteine, Methionine, Tryptophan, Alanine, Isoleucine, Leucine, Phenylalanine, Valine, Proline, and Glycine.

As used herein, a “non-natural amino acid side chain” refers to the side-chain substituent of a non-naturally occurring amino acid. Non-natural amino acids include β-amino acids (β³ and β²), Homo-amino acids, Proline and Pyruvic acid derivatives, 3-substituted Alanine derivatives, Glycine derivatives, Ring-substituted Phenylalanine and Tyrosine Derivatives, Linear core amino acids and N-methyl amino acids. Exemplary non-natural amino acids are available from Sigma-Aldridge, listed under “unnatural amino acids & derivatives.” See also, Travis S. Young and Peter G. Schultz, “Beyond the Canonical 20 Amino Acids: Expanding the Genetic Lexicon,” J. Biol. Chem. 2010 285: 11039-11044, which is incorporated by reference in its entirety.

As used herein, a substituted group is derived from the unsubstituted parent group in which there has been an exchange of one or more hydrogen atoms for another atom or group. Unless otherwise indicated, when a group is deemed to be “substituted,” it is meant that the group is substituted with one or more substituents independently selected from C₁-C₆ alkyl, C₁-C₆ alkenyl, C₁-C₆ alkynyl, C₁-C₆ heteroalkyl, C₃-C₁ carbocyclyl (optionally substituted with halo, C₁-C₆ alkyl, C₁-C₆ alkoxy, C₁-C₆ haloalkyl, and C₁-C₆ haloalkoxy), C₃-C₇-carbocyclyl-C₁-C₆-alkyl (optionally substituted with halo, C₁-C₆ alkyl, C₁-C₆ alkoxy, C₁-C₆ haloalkyl, and C₁-C₆ haloalkoxy), 5-10 membered heterocyclyl (optionally substituted with halo, C₁-C₆ alkyl, C₁-C₆ alkoxy, C₁-C₆ haloalkyl, and C₁-C₆ haloalkoxy), 5-10 membered heterocyclyl-C₁-C₆-alkyl (optionally substituted with halo, C₁-C₆ alkyl, C₁-C₆ alkoxy, C₁-C₆ haloalkyl, and C₁-C₆ haloalkoxy), aryl (optionally substituted with halo, C₁-C₆ alkyl, C₁-C₆ alkoxy, C₁-C₆ haloalkyl, and C₁-C₆ haloalkoxy), aryl(C₁-C₆)alkyl (optionally substituted with halo, C₁-C₆ alkyl, C₁-C₆ alkoxy, C₁-C₆ haloalkyl, and C₁-C₆ haloalkoxy), aryl(C₁-C₆)heteroalkyl (optionally substituted with halo, C₁-C₆ alkyl, C₁-C₆ alkoxy, C₁-C₆ haloalkyl, and C₁-C₆ haloalkoxy), 5-10 membered heteroaryl (optionally substituted with halo, C₁-C₆ alkyl, C₁-C₆ alkoxy, C₁-C₆ haloalkyl, and C₁-C₆ haloalkoxy), 5-10 membered heteroaryl(C₁-C₆)alkyl (optionally substituted with halo, C₁-C₆ alkyl, C₁-C₆ alkoxy, C₁-C₆ haloalkyl, and C₁-C₆ haloalkoxy), halo, cyano, hydroxy, C₁-C₆ alkoxy, C₁-C₆ alkoxy(C₁-C₆)alkyl (i.e., ether), aryloxy, sulfhydryl (mercapto), halo(C₁-C₆)alkyl (e.g., —CF₃), halo(C₁-C₆)alkoxy (e.g., —OCF₃), C₁-C₆ alkylthio, arylthio, amino, amino(C₁-C₆)alkyl, nitro, O-carbamyl, N-carbamyl, O-thiocarbamyl, N-thiocarbamyl, C-amido, N-amido, S-sulfonamido, N-sulfonamido, C-carboxy, O-carboxy, acyl, cyanato, isocyanato, thiocyanato, isothiocyanato, sulfinyl, sulfonyl, and oxo (═O). Wherever a group is described as “optionally substituted” that group can be substituted with the above substituents.

In some embodiments, substituted group(s) is (are) substituted with one or more substituent(s) individually and independently selected from C₁-C₄ alkyl, amino, hydroxy, and halogen.

It is to be understood that certain radical naming conventions can include either a mono-radical or a di-radical, depending on the context. For example, where a substituent requires two points of attachment to the rest of the molecule, it is understood that the substituent is a di-radical. For example, a substituent identified as alkyl that requires two points of attachment includes di-radicals such as —CH₂—, —CH₂CH₂—, —CH₂CH(CH₃)CH₂—, and the like. Other radical naming conventions clearly indicate that the radical is a di-radical such as “alkylene” or “alkenylene.”

When two R groups are said to form a ring (e.g., a carbocyclyl, heterocyclyl, aryl, or heteroaryl ring) “together with the atom to which they are attached,” it is meant that the collective unit of the atom and the two R groups are the recited ring. The ring is not otherwise limited by the definition of each R group when taken individually. For example, when the following substructure is present:

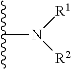

and R¹ and R² are defined as selected from the group consisting of hydrogen and alkyl, or R¹ and R² together with the nitrogen to which they are attached form a heterocyclyl, it is meant that R¹ and R² can be selected from hydrogen or alkyl, or alternatively, the substructure has structure:

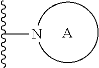

where ring A is a heterocyclyl ring containing the depicted nitrogen.

Similarly, when two “adjacent” R groups are said to form a ring “together with the atoms to which they are attached,” it is meant that the collective unit of the atoms, intervening bonds, and the two R groups are the recited ring. For example, when the following substructure is present:

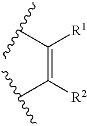

and R¹ and R² are defined as selected from the group consisting of hydrogen and alkyl, or R¹ and R² together with the atoms to which they are attached form an aryl or carbocyclyl, it is meant that R¹ and R² can be selected from hydrogen or alkyl, or alternatively, the substructure has structure:

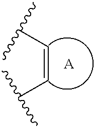

where A is an aryl ring or a carbocyclyl containing the depicted double bond.

Wherever a substituent is depicted as a di-radical (i.e., has two points of attachment to the rest of the molecule), it is to be understood that the substituent can be attached in any directional configuration unless otherwise indicated. Thus, for example, a substituent depicted as -AE- or

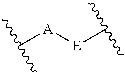

includes the substituent being oriented such that the A is attached at the leftmost attachment point of the molecule as well as the case in which A is attached at the rightmost attachment point of the molecule.

The term “agent” or “test agent” includes any substance, molecule, element, compound, entity, or a combination thereof. It includes, but is not limited to, e.g., protein, polypeptide, peptide or mimetic, small organic molecule, polysaccharide, polynucleotide, and the like. It can be a natural product, a synthetic compound, or a chemical compound, or a combination of two or more substances. Unless otherwise specified, the terms “agent”, “substance”, and “compound” are used interchangeably herein.

The term “analog” is used herein to refer to a molecule that structurally resembles a reference molecule but which has been modified in a targeted and controlled manner, by replacing a specific substituent of the reference molecule with an alternate substituent. Compared to the reference molecule, an analog would be expected, by one skilled in the art, to exhibit the same, similar, or improved utility. Synthesis and screening of analogs, to identify variants of known compounds having improved characteristics (such as higher binding affinity for a target molecule) is an approach that is well known in pharmaceutical chemistry.

The term “mammal” is used in its usual biological sense. Thus, it specifically includes, but is not limited to, primates, including simians (chimpanzees, apes, monkeys) and humans, cattle, horses, sheep, goats, swine, rabbits, dogs, cats, rats and mice but also includes many other species.

The term “microbial infection” refers to the invasion of the host organism, whether the organism is a vertebrate, invertebrate, fish, plant, bird, or mammal, by pathogenic microbes. This includes the excessive growth of microbes that are normally present in or on the body of a mammal or other organism. More generally, a microbial infection can be any situation in which the presence of a microbial population(s) is damaging to a host mammal. Thus, a mammal is “suffering” from a microbial infection when excessive numbers of a microbial population are present in or on a mammal's body, or when the effects of the presence of a microbial population(s) is damaging the cells or other tissue of a mammal. Specifically, this description applies to a bacterial infection. Note that the compounds of preferred embodiments are also useful in treating microbial growth or contamination of cell cultures or other media, or inanimate surfaces or objects, and nothing herein should limit the preferred embodiments only to treatment of higher organisms, except when explicitly so specified in the claims.

The term “pharmaceutically acceptable carrier” or “pharmaceutically acceptable excipient” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the active ingredient, its use in the therapeutic compositions is contemplated. In addition, various adjuvants such as are commonly used in the art may be included. Considerations for the inclusion of various components in pharmaceutical compositions are described, e.g., in Gilman et al. (Eds.) (1990); Goodman and Gilman's: The Pharmacological Basis of Therapeutics, 8th Ed., Pergamon Press, which is incorporated herein by reference in its entirety.

“Subject” as used herein, means a human or a non-human mammal, e.g., a dog, a cat, a mouse, a rat, a cow, a sheep, a pig, a goat, a non-human primate or a bird, e.g., a chicken, as well as any other vertebrate or invertebrate.

An “effective amount” or a “therapeutically effective amount” as used herein refers to an amount of a therapeutic agent that is effective to relieve, to some extent, or to reduce the likelihood of onset of, one or more of the symptoms of a disease or condition, and includes curing a disease or condition. “Curing” means that the symptoms of a disease or condition are eliminated; however, certain long-term or permanent effects may exist even after a cure is obtained (such as extensive tissue damage).

“Treat,” “treatment,” or “treating,” as used herein refers to administering a pharmaceutical composition for prophylactic and/or therapeutic purposes. The term “prophylactic treatment” refers to treating a subject who does not yet exhibit symptoms of a disease or condition, but who is susceptible to, or otherwise at risk of, a particular disease or condition, whereby the treatment reduces the likelihood that the patient will develop the disease or condition.

### Methods of Preparation

The compounds disclosed herein may be synthesized by methods described below, or by modification of these methods. Ways of modifying the methodology include, among others, temperature, solvent, reagents etc., known to those skilled in the art. In general, during any of the processes for preparation of the compounds disclosed herein, it may be necessary and/or desirable to protect sensitive or reactive groups on any of the molecules concerned. This may be achieved by means of conventional protecting groups, such as those described in Protective Groups in Organic Chemistry (ed. J. F. W. McOmie, Plenum Press, 1973); and P. G. M. Green, T. W. Wutts, Protecting Groups in Organic Synthesis (3rd ed.) Wiley, New York (1999), which are both hereby incorporated herein by reference in their entirety. The protecting groups may be removed at a convenient subsequent stage using methods known from the art. Synthetic chemistry transformations useful in synthesizing applicable compounds are known in the art and include e.g. those described in R. Larock, Comprehensive Organic Transformations, VCH Publishers, 1989, or L. Paquette, ed., Encyclopedia of Reagents for Organic Synthesis, John Wiley and Sons, 1995, which are both hereby incorporated herein by reference in their entirety. The routes shown and described herein are illustrative only and are not intended, nor are they to be construed, to limit the scope of the claims in any manner whatsoever. Those skilled in the art will be able to recognize modifications of the disclosed syntheses and to devise alternate routes based on the disclosures herein; all such modifications and alternate routes are within the scope of the claims.

In the following schemes, protecting groups for oxygen atoms are selected for their compatibility with the requisite synthetic steps as well as compatibility of the introduction and deprotection steps with the overall synthetic schemes (P. G. M. Green, T. W. Wutts, Protecting Groups in Organic Synthesis (3rd ed.) Wiley, New York (1999)).

If the compounds of the present technology contain one or more chiral centers, such compounds can be prepared or isolated as pure stereoisomers, i.e., as individual enantiomers or d(l) stereoisomers, or as stereoisomer-enriched mixtures. All such stereoisomers (and enriched mixtures) are included within the scope of the present technology, unless otherwise indicated. Pure stereoisomers (or enriched mixtures) may be prepared using, for example, optically active starting materials or stereoselective reagents well-known in the art. Alternatively, racemic mixtures of such compounds can be separated using, for example, chiral column chromatography, chiral resolving agents and the like.

The starting materials for the following reactions are generally known compounds or can be prepared by known procedures or obvious modifications thereof. For example, many of the starting materials are available from commercial suppliers such as Aldrich Chemical Co. (Milwaukee, Wis., USA), Bachem (Torrance, Calif., USA), Emka-Chemce or Sigma (St. Louis, Mo., USA). Others may be prepared by procedures, or obvious modifications thereof, described in standard reference texts such as Fieser and Fieser's Reagents for Organic Synthesis, Volumes 1-15 (John Wiley, and Sons, 1991), Rodd's Chemistry of Carbon Compounds, Volumes 1-5, and Supplementals (Elsevier Science Publishers, 1989), Organic Reactions, Volumes 1-40 (John Wiley, and Sons, 1991), March's Advanced Organic Chemistry, (John Wiley, and Sons, 5th Edition, 2001), and Larock's Comprehensive Organic Transformations (VCH Publishers Inc., 1989).

### Administration and Pharmaceutical Compositions

Administration of the compounds disclosed herein, or the pharmaceutically acceptable salts thereof can be via any of the accepted modes of administration for agents that serve similar utilities including, but not limited to, orally, subcutaneously, intravenously, intranasally, topically, transdermally, intraperitoneally, intramuscularly, intrapulmonarilly, vaginally, rectally, or intraocularly. Oral and parenteral administrations are customary in treating the indications that are the subject of the preferred embodiments.

The compounds useful as described above can be formulated into pharmaceutical compositions for use in treatment of these conditions. Standard pharmaceutical formulation techniques are used, such as those disclosed in Remington's The Science and Practice of Pharmacy, 21st Ed., Lippincott Williams & Wilkins (2005), incorporated by reference in its entirety. Accordingly, some embodiments include pharmaceutical compositions comprising: (a) a safe and therapeutically effective amount of a compound described herein (including enantiomers, diastereoisomers, tautomers, polymorphs, and solvates thereof), or pharmaceutically acceptable salts thereof; and (b) a pharmaceutically acceptable carrier, diluent, excipient or combination thereof.

In addition to the selected compound useful as described above, some embodiments include compositions containing a pharmaceutically acceptable carrier. The term “pharmaceutically acceptable carrier” or “pharmaceutically acceptable excipient” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the active ingredient, its use in the therapeutic compositions is contemplated. In addition, various adjuvants such as are commonly used in the art may be included. Considerations for the inclusion of various components in pharmaceutical compositions are described, e.g., in Gilman et al. (Eds.) (1990); Goodman and Gilman's: The Pharmacological Basis of Therapeutics, 8th Ed., Pergamon Press, which is incorporated herein by reference in its entirety.

Some examples of substances, which can serve as pharmaceutically-acceptable carriers or components thereof, are sugars, such as lactose, glucose and sucrose; starches, such as corn starch and potato starch; cellulose and its derivatives, such as sodium carboxymethyl cellulose, ethyl cellulose, and methyl cellulose; powdered tragacanth; malt; gelatin; talc; solid lubricants, such as stearic acid and magnesium stearate; calcium sulfate; vegetable oils, such as peanut oil, cottonseed oil, sesame oil, olive oil, corn oil and oil of theobroma; polyols such as propylene glycol, glycerine, sorbitol, mannitol, and polyethylene glycol; alginic acid; emulsifiers, such as the TWEENS; wetting agents, such sodium lauryl sulfate; coloring agents; flavoring agents; tableting agents, stabilizers; antioxidants; preservatives; pyrogen-free water; isotonic saline; and phosphate buffer solutions.

The choice of a pharmaceutically acceptable carrier to be used in conjunction with the subject compound is basically determined by the way the compound is to be administered.

The compositions described herein are preferably provided in unit dosage form. As used herein, a “unit dosage form” is a composition containing an amount of a compound that is suitable for administration to an animal, preferably mammal subject, in a single dose, according to good medical practice. The preparation of a single or unit dosage form, however, does not imply that the dosage form is administered once per day or once per course of therapy. Such dosage forms are contemplated to be administered once, twice, thrice or more per day and may be administered as infusion over a period of time (e.g., from about 30 minutes to about 2-6 hours), or administered as a continuous infusion, and may be given more than once during a course of therapy, though a single administration is not specifically excluded. The skilled artisan will recognize that the formulation does not specifically contemplate the entire course of therapy and such decisions are left for those skilled in the art of treatment rather than formulation.

The compositions useful as described above may be in any of a variety of suitable forms for a variety of routes for administration, for example, for oral, nasal, rectal, topical (including transdermal), ocular, intracerebral, intracranial, intrathecal, intra-arterial, intravenous, intramuscular, or other parental routes of administration. The skilled artisan will appreciate that oral and nasal compositions comprise compositions that are administered by inhalation, and made using available methodologies. Depending upon the particular route of administration desired, a variety of pharmaceutically acceptable carriers well-known in the art may be used. Pharmaceutically acceptable carriers include, for example, solid or liquid fillers, diluents, hydrotropies, surface-active agents, and encapsulating substances. Optional pharmaceutically active materials may be included, which do not substantially interfere with the inhibitory activity of the compound. The amount of carrier employed in conjunction with the compound is sufficient to provide a practical quantity of material for administration per unit dose of the compound. Techniques and compositions for making dosage forms useful in the methods described herein are described in the following references, all incorporated by reference herein: Modern Pharmaceutics, 4th Ed., Chapters 9 and 10 (Banker & Rhodes, editors, 2002); Lieberman et al., Pharmaceutical Dosage Forms: Tablets (1989); and Ansel, Introduction to Pharmaceutical Dosage Forms 8th Edition (2004).

Various oral dosage forms can be used, including such solid forms as tablets, capsules, granules and bulk powders. Tablets can be compressed, tablet triturates, enteric-coated, sugar-coated, film-coated, or multiple-compressed, containing suitable binders, lubricants, diluents, disintegrating agents, coloring agents, flavoring agents, flow-inducing agents, and melting agents. Liquid oral dosage forms include aqueous solutions, emulsions, suspensions, solutions and/or suspensions reconstituted from non-effervescent granules, and effervescent preparations reconstituted from effervescent granules, containing suitable solvents, preservatives, emulsifying agents, suspending agents, diluents, sweeteners, melting agents, coloring agents and flavoring agents.

The pharmaceutically acceptable carrier suitable for the preparation of unit dosage forms for peroral administration is well-known in the art. Tablets typically comprise conventional pharmaceutically compatible adjuvants as inert diluents, such as calcium carbonate, sodium carbonate, mannitol, lactose, and cellulose; binders such as starch, gelatin, and sucrose; disintegrants such as starch, alginic acid and croscarmelose; lubricants such as magnesium stearate, stearic acid, and talc. Glidants such as silicon dioxide can be used to improve flow characteristics of the powder mixture. Coloring agents, such as the FD&C dyes, can be added for appearance. Sweeteners and flavoring agents, such as aspartame, saccharin, menthol, peppermint, and fruit flavors, are useful adjuvants for chewable tablets. Capsules typically comprise one or more solid diluents disclosed above. The selection of carrier components depends on secondary considerations like taste, cost, and shelf stability, which are not critical, and can be readily made by a person skilled in the art.

Peroral compositions also include liquid solutions, emulsions, suspensions, and the like. The pharmaceutically acceptable carriers suitable for preparation of such compositions are well known in the art. Typical components of carriers for syrups, elixirs, emulsions and suspensions include ethanol, glycerol, propylene glycol, polyethylene glycol, liquid sucrose, sorbitol and water. For a suspension, typical suspending agents include methyl cellulose, sodium carboxymethyl cellulose, AVICEL RC-591, tragacanth and sodium alginate; typical wetting agents include lecithin and polysorbate 80; and typical preservatives include methyl paraben and sodium benzoate. Peroral liquid compositions may also contain one or more components such as sweeteners, flavoring agents and colorants disclosed above.

Such compositions may also be coated by conventional methods, typically with pH or time-dependent coatings, such that the subject compound is released in the gastrointestinal tract in the vicinity of the desired topical application, or at various times to extend the desired action. Such dosage forms typically include, but are not limited to, one or more of cellulose acetate phthalate, polyvinylacetate phthalate, hydroxypropyl methyl cellulose phthalate, ethyl cellulose, Eudragit coatings, waxes and shellac.

Compositions described herein may optionally include other drug actives.

Other compositions useful for attaining systemic delivery of the subject compounds include sublingual, buccal and nasal dosage forms. Such compositions typically comprise one or more of soluble filler substances such as sucrose, sorbitol and mannitol; and binders such as acacia, microcrystalline cellulose, carboxymethyl cellulose and hydroxypropyl methyl cellulose. Glidants, lubricants, sweeteners, colorants, antioxidants and flavoring agents disclosed above may also be included.

A liquid composition, which is formulated for topical ophthalmic use, is formulated such that it can be administered topically to the eye. The comfort should be maximized as much as possible, although sometimes formulation considerations (e.g. drug stability) may necessitate less than optimal comfort. In the case that comfort cannot be maximized, the liquid should be formulated such that the liquid is tolerable to the patient for topical ophthalmic use. Additionally, an ophthalmically acceptable liquid should either be packaged for single use, or contain a preservative to prevent contamination over multiple uses.

For ophthalmic application, solutions or medicaments are often prepared using a physiological saline solution as a major vehicle. Ophthalmic solutions should preferably be maintained at a comfortable pH with an appropriate buffer system. The formulations may also contain conventional, pharmaceutically acceptable preservatives, stabilizers and surfactants.

Preservatives that may be used in the pharmaceutical compositions disclosed herein include, but are not limited to, benzalkonium chloride, PHMB, chlorobutanol, thimerosal, phenylmercuric, acetate and phenylmercuric nitrate. A useful surfactant is, for example, Tween 80. Likewise, various useful vehicles may be used in the ophthalmic preparations disclosed herein. These vehicles include, but are not limited to, polyvinyl alcohol, povidone, hydroxypropyl methyl cellulose, poloxamers, carboxymethyl cellulose, hydroxyethyl cellulose and purified water.

Tonicity adjustors may be added as needed or convenient. They include, but are not limited to, salts, particularly sodium chloride, potassium chloride, mannitol and glycerin, or any other suitable ophthalmically acceptable tonicity adjustor.

Various buffers and means for adjusting pH may be used so long as the resulting preparation is ophthalmically acceptable. For many compositions, the pH will be between 4 and 9. Accordingly, buffers include acetate buffers, citrate buffers, phosphate buffers and borate buffers. Acids or bases may be used to adjust the pH of these formulations as needed.

In a similar vein, an ophthalmically acceptable antioxidant includes, but is not limited to, sodium metabisulfite, sodium thiosulfate, acetylcysteine, butylated hydroxyanisole and butylated hydroxytoluene.

Other excipient components, which may be included in the ophthalmic preparations, are chelating agents. A useful chelating agent is edetate disodium, although other chelating agents may also be used in place or in conjunction with it.

For topical use, creams, ointments, gels, solutions or suspensions, etc., containing the compound disclosed herein are employed. Topical formulations may generally be comprised of a pharmaceutical carrier, co-solvent, emulsifier, penetration enhancer, preservative system, and emollient.

For intravenous administration, the compounds and compositions described herein may be dissolved or dispersed in a pharmaceutically acceptable diluent, such as a saline or dextrose solution. Suitable excipients may be included to achieve the desired pH, including but not limited to NaOH, sodium carbonate, sodium acetate, HCl, and citric acid. In various embodiments, the pH of the final composition ranges from 2 to 8, or preferably from 4 to 7. Antioxidant excipients may include sodium bisulfite, acetone sodium bisulfite, sodium formaldehyde, sulfoxylate, thiourea, and EDTA. Other non-limiting examples of suitable excipients found in the final intravenous composition may include sodium or potassium phosphates, citric acid, tartaric acid, gelatin, and carbohydrates such as dextrose, mannitol, and dextran. Further acceptable excipients are described in Powell, et al., Compendium of Excipients for Parenteral Formulations, PDA J Pharm Sci and Tech 1998, 52 238-311 and Nema et al., Excipients and Their Role in Approved Injectable Products: Current Usage and Future Directions, PDA J Pharm Sci and Tech 2011, 65 287-332, both of which are incorporated herein by reference in their entirety. Antimicrobial agents may also be included to achieve a bacteriostatic or fungistatic solution, including but not limited to phenylmercuric nitrate, thimerosal, benzethonium chloride, benzalkonium chloride, phenol, cresol, and chlorobutanol.

The compositions for intravenous administration may be provided to caregivers in the form of one more solids that are reconstituted with a suitable diluent such as sterile water, saline or dextrose in water shortly prior to administration. In other embodiments, the compositions are provided in solution ready to administer parenterally. In still other embodiments, the compositions are provided in a solution that is further diluted prior to administration. In embodiments that include administering a combination of a compound described herein and another agent, the combination may be provided to caregivers as a mixture, or the caregivers may mix the two agents prior to administration, or the two agents may be administered separately.

The actual dose of the active compounds described herein depends on the specific compound, and on the condition to be treated; the selection of the appropriate dose is well within the knowledge of the skilled artisan.

The compounds and compositions described herein, if desired, may be presented in a pack or dispenser device containing one or more unit dosage forms containing the active ingredient. Such a pack or device may, for example, comprise metal or plastic foil, such as a blister pack, or glass, and rubber stoppers such as in vials. The pack or dispenser device may be accompanied by instructions for administration. Compounds and compositions described herein are formulated in a compatible pharmaceutical carrier may also be prepared, placed in an appropriate container, and labeled for treatment of an indicated condition.

The amount of the compound in a formulation can vary within the full range employed by those skilled in the art. Typically, the formulation will contain, on a weight percent (wt %) basis, from about 0.01 99.99 wt % of a compound of the present technology based on the total formulation, with the balance being one or more suitable pharmaceutical excipients. Preferably, the compound is present at a level of about 1 80 wt %.

### Methods of Treatment

Some embodiments described herein relate to a method of treating a cancer, comprising administering to a subject in need thereof, a compound as described herein. In some embodiments, the cancer is an oncogenic Ras-mediated cancer. In some embodiments, the Ras-mediated cancer is pancreatic cancer or acute myeloid leukemia. In some embodiments, the concentration in the subject upon administration is no greater than about 500 μM. In other embodiments, the concentration in the subject upon administration is no greater than about 100 μM. In further embodiments, the concentration in the subject upon administration is no greater than about 50 μM. In some embodiments, the concentration in the subject upon administration is no greater than about 40 μM. In some embodiments, the concentration in the subject upon administration is no greater than about 10 μM. In some embodiments, the concentration in the subject upon administration is no greater than about 1 μM.

Some embodiments of the present invention include methods of treating oncogenic Ras-mediated cancers with the compounds and compositions comprising the compounds described herein. Some methods include administering a compound, composition, pharmaceutical composition described herein to a subject in need thereof. In some embodiments, a subject can be an animal, e.g., a mammal (including a human). As will be appreciated from the foregoing, methods of treating a cancer include methods for preventing cancer in a subject at risk thereof.

In some embodiments, the subject is a human.

Further embodiments include administering a combination of compounds to a subject in need thereof. A combination can include a compound, composition, pharmaceutical composition described herein with an additional medicament.

Some embodiments include co-administering a compound, composition, and/or pharmaceutical composition described herein, with an additional medicament. By “co-administration,” it is meant that the two or more agents may be found in the patient's bloodstream at the same time, regardless of when or how they are actually administered. In one embodiment, the agents are administered simultaneously. In one such embodiment, administration in combination is accomplished by combining the agents in a single dosage form. In another embodiment, the agents are administered sequentially. In one embodiment, the agents are administered through the same route, such as orally. In another embodiment, the agents are administered through different routes, such as one being administered orally and another being administered intravenously.

Examples of additional medicaments include an antibacterial agent, antifungal agent, an antiviral agent, an anti-inflammatory agent and an anti-allergic agent.

### Screening

Some embodiments described herein relate to a method of testing a compound for an ability to inhibit a Ras-mediated cancer comprising: contacting a Ras-mediated cancer cell culture with a test compound; determining the percent cell survival of the cells within the Ras-mediated cancer cell culture; identifying a compound as having an ability to inhibit a Ras-mediated cancer when the percent cell survival of the cells within the Ras-mediated cancer cell culture is below a threshold amount.

Additional embodiments described herein relate to a method of testing a compound for an ability to inhibit an oncogenic Ras comprising: contacting a Ras-mediated cancer cell culture with a test compound; determining the percent cell survival of the cells within the Ras-mediated cancer cell culture; identifying a compound as having an ability to inhibit a Ras-mediated cancer when the percent cell survival of the cells within the Ras-mediated cancer cell culture is below a threshold amount.

Further embodiments described herein relate to a method of testing a compound for an ability to bind an allosteric inhibition site of SOS1 comprising: contacting a Ras-mediated cancer cell culture with a test compound; determining the percent cell survival of the cells within the Ras-mediated cancer cell culture; identifying a compound as having an ability to bind an allosteric inhibition site of SOS1 when the percent cell survival of the cells within the Ras-mediated cancer cell culture is below a threshold amount.

In some embodiments, the compound binds to the allosteric site of SOS1. In some embodiments, the threshold amount for the percent cell survival of the cells within the Ras-mediated cancer cell culture is the percent cell survival of the cells within the Ras-mediated cancer cell culture when contacted with NSC-70220. In some embodiments, the threshold amount for the percent cell survival of the cells within the Ras-mediated cancer cell culture is 10-50 percent. In some embodiments, the threshold amount for the percent cell survival of the cells within the Ras-mediated cancer cell culture is 10-20 percent. In some embodiments, the threshold amount for the percent cell survival of the cells within the Ras-mediated cancer cell culture is 20 percent or less. In some embodiments, the threshold amount for the percent cell survival of the cells within the Ras-mediated cancer cell culture is 10 percent or less. In some embodiments, the threshold amount for the percent cell survival of the cells within the Ras-mediated cancer cell culture is 5 percent or less. In some embodiments, the threshold amount for the percent cell survival of the cells within the Ras-mediated cancer cell culture is 1 percent or less.

Some embodiments relate to a method further comprising: contacting a wild-type cell culture with a test compound; determining the percent cell survival of the cells within the wild-type cell culture; identifying a compound as specifically binding the allosteric inhibition site of SOS1 when the percent cell survival of the cells within the wild-type cell culture is above a threshold amount.

In some embodiments, the threshold amount for the percent cell survival of the cells within the wild-type cell culture is 10-90 percent. In some embodiments, the threshold amount for the percent cell survival of the cells within the wild-type cell culture is 20-80 percent. In some embodiments, the threshold amount for the percent cell survival of the cells within the wild-type cell culture is 30-70 percent. In some embodiments, the threshold amount for the percent cell survival of the cells within the wild-type cell culture is 40-60 percent.

In some embodiments, the threshold amount for the percent cell survival of the cells within the wild-type cell culture is 50 percent or more. In some embodiments, the threshold amount for the percent cell survival of the cells within the wild-type cell culture is 60 percent or more. In some embodiments, the wild-type cell culture comprises the same cell type as the Ras-mediated cancer cell culture. For example, the cell type would be a pancreatic cell or a myeloid blood cell, where the Ras-mediated cancer cell would respectively be a pancreatic cancer cell or an acute myeloid leukemia cell, and the respective wild type cell would be a wild type pancreatic cell or a wild type myeloid blood cell. In some embodiments, the wild-type cell culture differs from the Ras-mediated cancer cell culture by virtue of the cells in the wild-type cell culture comprising wild-type Ras and the cells in the Ras-mediated cancer cell culture comprising oncogenic Ras. In some embodiments, the Ras-mediated cancer is pancreatic cancer or acute myeloid leukemia.

In further embodiments, the concentration of the compound is about 1 μM to about 500 μM. In some embodiments, the concentration of the compound is about 10 μM to about 50 μM.

In some embodiments, the percent cell survival of the cells within the Ras-mediated cancer cell culture is the percent cell survival after 1-7 days. In some embodiments, the percent cell survival of the cells within the Ras-mediated cancer cell culture is the percent cell survival after 2-5 days. In some embodiments, the percent cell survival of the cells within the Ras-mediated cancer cell culture is the percent cell survival after 3-4 days. In some embodiments, the percent cell survival of the cells within the Ras-mediated cancer cell culture is the percent cell survival after 1, 2, 3, 4, 5, 6, 7, 8, 9, 10, 11, 12, 13, 14, 15, 16, 17, 18, 19, or 20 days. In some embodiments, the percent cell survival of the cells within the Ras-mediated cancer cell culture is the percent cell survival after about 2 days. In some embodiments, the percent cell survival of the cells within the Ras-mediated cancer cell culture is the percent cell survival after 1, 2, 3, 4, 5, 6, 7, 8, 9, 10, 11, 12, 13, 14, 15, 16, 17, 18, 19, 20, 21, 22, or 23, hours.

In some embodiments, the percent cell survival of the cells within the wild-type cell culture is the percent cell survival after 2-5 days. In some embodiments, the percent cell survival of the cells within the wild-type cell culture is the percent cell survival after 3-4 days. In some embodiments, the percent cell survival of the cells within the wild-type cell culture is the percent cell survival after 1, 2, 3, 4, 5, 6, 7, 8, 9, 10, 11, 12, 13, 14, 15, 16, 17, 18, 19, or 20 days. In some embodiments, the percent cell survival of the cells within the wild-type cell culture is the percent cell survival after about 2 days. In some embodiments, the percent cell survival of the cells within the wild-type cell culture is the percent cell survival after 1, 2, 3, 4, 5, 6, 7, 8, 9, 10, 11, 12, 13, 14, 15, 16, 17, 18, 19, 20, 21, 22, or 23, hours.

In certain embodiments, methods of screening a small molecule compound library using in silico docking followed by experimental testing can be used to identify compounds that target SOS1. In certain embodiments, the compound library includes small molecule compounds that inhibit SOS1 by targeting the allosteric site.

A multistep docking scheme may involve screening small molecules with both the bound (PDB ID: 1XD2) and unbound SOS1 (PDB ID: 1XD4) crystal structures targeting the allosteric site. Once the in silico screen identifies candidate compounds, some these compounds may be selected for experimental testing based upon compound chemical space dissimilarities.

In some embodiments, the compounds are selected for the ability to bind the allosteric site of SOS1.

The experimental screening may feature fluorescence-based guanine nucleotide exchange dissociation assays. This screening for inhibition of active Ras-stimulated GEF activity toward Ras-GTP loading may further identify effective compounds which may be subject to further biochemical validation. Then, a SOS1 catalytic site binding deficient mutant, such as Ras(Y64A), may be used to show which compounds are capable of dose-dependently inhibiting constitutively active Ras(Y64A):GTPγS-enhanced allosteric activation of SOS1_{cat} toward WT Ras-GDP (allosteric effect), but not SOS1_{cat}-mediated catalytic activation of WT Ras-GDP in the absence of Ras(Y64A):GTPγS (catalytic activity). These methods provide a virtual screening coupled with experimental screening system for selectively identifying SOS1 allosteric site inhibitors.

### EXAMPLES

### Activity Screen

Mononuclear bone marrow cells harvested from Kras G12D/+ mice were tested for proliferation in a CellTiter-Glo® Luminescent Cell Viability Assay by Promega (Madison, Wis.) carried out by following the instructions provided by the manufacturer in the presence of 40 μM of 176 selected test compounds. A few of the tested compounds demonstrated superior growth inhibitory activity. The structures of these active compounds are shown in Table 1, along with the percentage of residual activity after two days of incubation with 40 μM of the compound. The residual activity is the percentage of cells that were still alive at the end of the experiment.

TABLE 1
Results from growth inhibitory assay.

| | | 40 μM |
|---|---|---|
| Compound | | (% Residual |
| Number | Structure | Activity) |
| NSC-70220 | 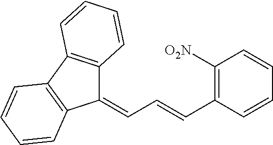 [SMILES: O=[N+]([O-])c1ccccc1/C=C/C=C1c2ccccc2-c2ccccc21] | 12 to 15 |
| 775432 | 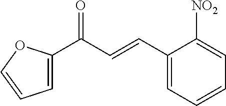 [SMILES: O=C(/C=C/c1ccccc1[N+](=O)[O-])c1ccco1] | 0 |
| 511128 | 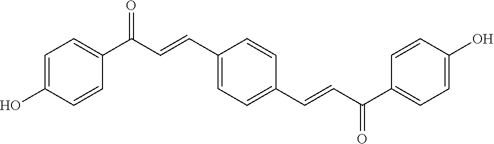 [SMILES: O=C(/C=C/c1ccc(/C=C/C(=O)c2ccc(O)cc2)cc1)c1ccc(O)cc1] | 1 |
| 388883 | 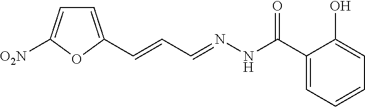 [SMILES: O=C(N/N=C/C=C/c1ccc([N+](=O)[O-])o1)c1ccccc1O] | 1 |
| 290794 | 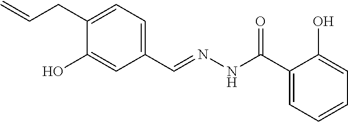 [SMILES: C=CCc1ccc(/C=N/NC(=O)c2ccccc2O)cc1O] | 2 |
| 394392 | 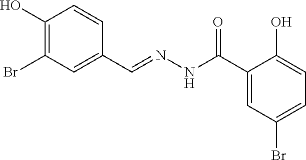 [SMILES: O=C(N/N=C/c1ccc(O)c(Br)c1)c1cc(Br)ccc1O] | 3 |
| 127987 | 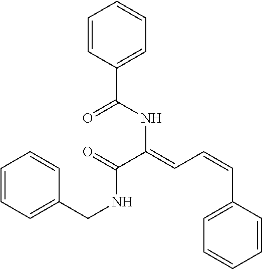 [SMILES: O=C(NCc1ccccc1)/C(=C/C=C\c1ccccc1)NC(=O)c1ccccc1] | 5 |
| 377089 | 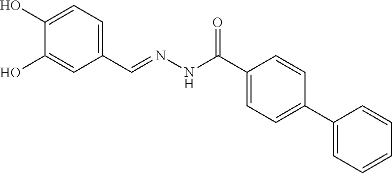 [SMILES: O=C(N/N=C/c1ccc(O)c(O)c1)c1ccc(-c2ccccc2)cc1] | 6 |
| 773778 | 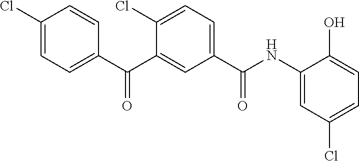 [SMILES: O=C(Nc1cc(Cl)ccc1O)c1ccc(Cl)c(C(=O)c2ccc(Cl)cc2)c1] | 8 |
| 312987 | 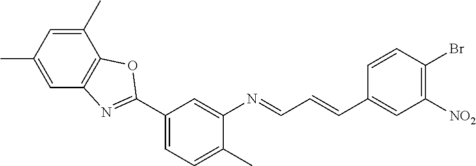 [SMILES: Cc1cc(C)c2oc(-c3ccc(C)c(/N=C/C=C/c4ccc(Br)c([N+](=O)[O-])c4)c3)nc2c1] | 8 |
| 387846 | 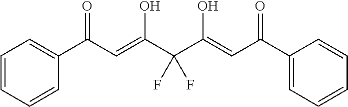 [SMILES: O=C(/C=C(\O)C(F)(F)/C(O)=C/C(=O)c1ccccc1)c1ccccc1] | 9 |
| 390481 | 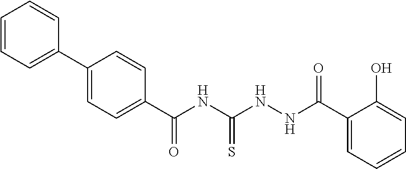 [SMILES: O=C(NC(=S)NNC(=O)c1ccccc1O)c1ccc(-c2ccccc2)cc1] | 10 |
| 377247 | 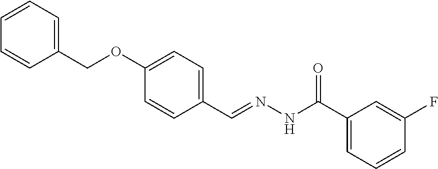 [SMILES: O=C(N/N=C/c1ccc(OCc2ccccc2)cc1)c1cccc(F)c1] | 11 |
| 393689 | 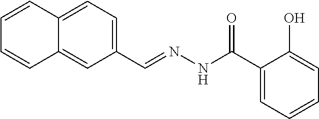 [SMILES: O=C(N/N=C/c1ccc2ccccc2c1)c1ccccc1O] | 11 |
| 408776 | 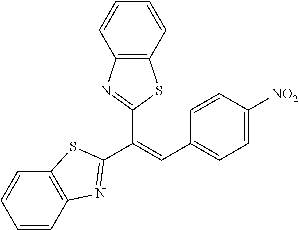 [SMILES: O=[N+]([O-])c1ccc(C=C(c2nc3ccccc3s2)c2nc3ccccc3s2)cc1] | 12 |
| 121366 | 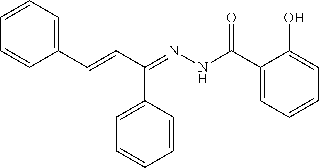 [SMILES: O=C(N/N=C(/C=C/c1ccccc1)c1ccccc1)c1ccccc1O] | 12 |
| 920300 | 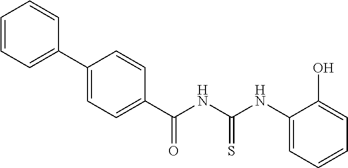 [SMILES: O=C(NC(=S)Nc1ccccc1O)c1ccc(-c2ccccc2)cc1] | 12 |
| 262294 | 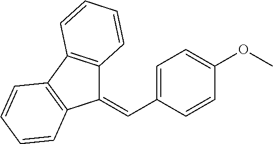 [SMILES: COc1ccc(C=C2c3ccccc3-c3ccccc32)cc1] | 13 |
| 473089 | 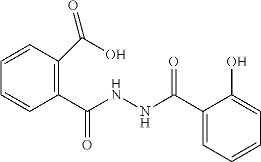 [SMILES: O=C(NNC(=O)c1ccccc1C(=O)O)c1ccccc1O] | 13 |
| 526470 | 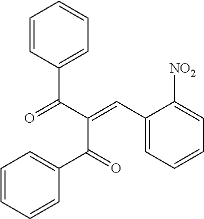 [SMILES: O=C(C(=Cc1ccccc1[N+](=O)[O-])C(=O)c1ccccc1)c1ccccc1] | 15 |
| 114395 | 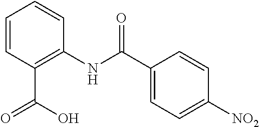 [SMILES: O=C(Nc1ccccc1C(=O)O)c1ccc([N+](=O)[O-])cc1] | 16 |

### Inhibition Studies

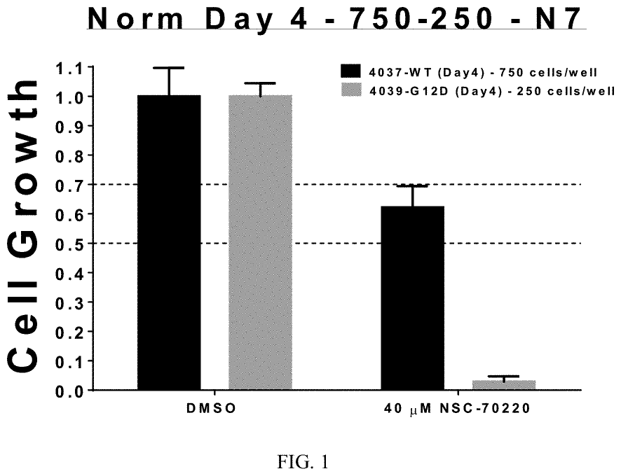

Kras (G12D) transformed pancreatic cancer cells (4039) and wild-type Kras expressing pancreatic cancer cells (4037) were each exposed to 40 μM of NSC-70220. Cell growth in the tissue culture was monitored by assays over a four-day period. The results of this experiment are shown in FIG. 1. The data show that at 40 μM, NSC-70220 selectively inhibits Kras (G12D) transformed pancreatic cancer cells (4039), but not wild-type Kras expressing pancreatic cancer cells (4037).

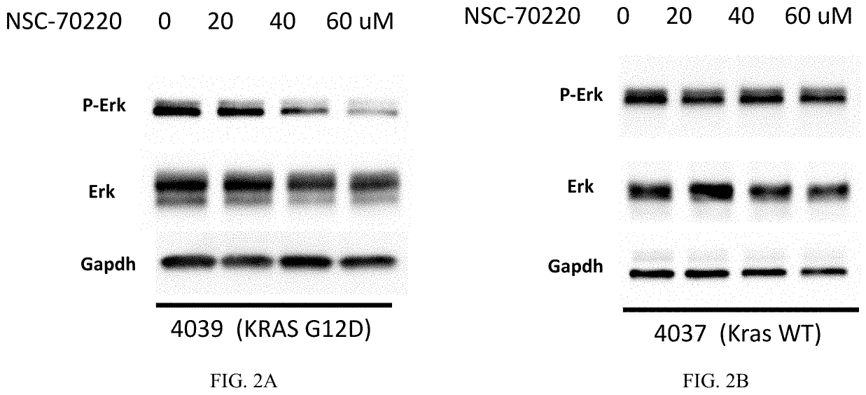

FIGS. 2A and 2B show that NSC-70220 selectively inhibits ERK activation of oncogenic Kras (G12D) pancreatic cancer cells but not the wild-type Kras pancreatic cells as assayed by Western blotting of p-ERK, total ERK, and loading control Gapdh.

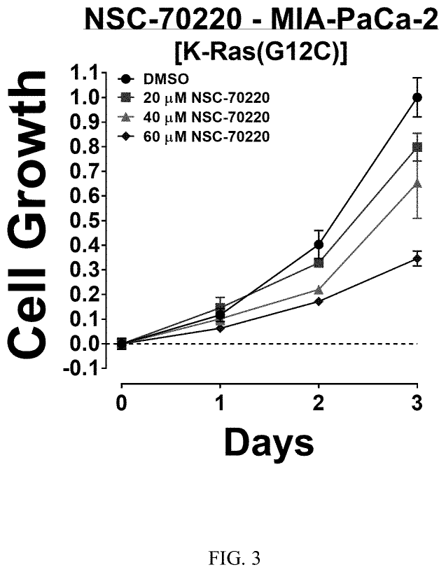

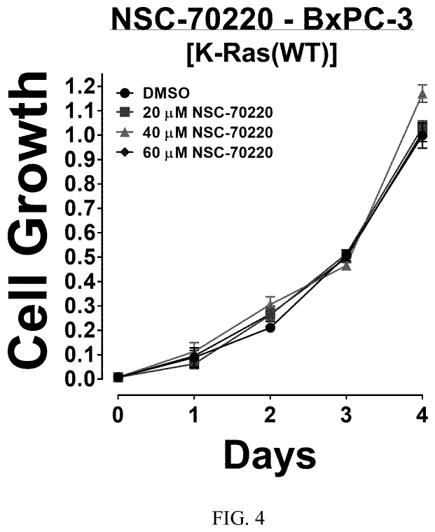

FIGS. 3-5 show data from experiments in which oncogenic Kras (G12C) transformed pancreatic MIA-PaCa-2 cells were treated with NSC-70220, and an experiment in which wild-type Kras expressing pancreatic BxPC-3 ductal cells were treated with NSC-70220. As shown in the graphs, NSC-70220 selectively inhibits growth of oncogenic Kras (G12C) transformed pancreatic cancer MIA-PaCa-2 cells, but not the wild-type Kras expressing pancreatic BxPC-3 ductal cells.

While some embodiments have been illustrated and described, a person with ordinary skill in the art, after reading the foregoing specification, can effect changes, substitutions of equivalents and other types of alterations to the compounds of the present technology or salts, pharmaceutical compositions, derivatives, prodrugs, metabolites, tautomers or racemic mixtures thereof as set forth herein. Each aspect and embodiment described above can also have included or incorporated therewith such variations or aspects as disclosed in regard to any or all of the other aspects and embodiments.

The present technology is also not to be limited in terms of the particular aspects described herein, which are intended as single illustrations of individual aspects of the present technology. Many modifications and variations of this present technology can be made without departing from its spirit and scope, as will be apparent to those skilled in the art. Functionally equivalent methods within the scope of the present technology, in addition to those enumerated herein, will be apparent to those skilled in the art from the foregoing descriptions. Such modifications and variations are intended to fall within the scope of the appended claims. It is to be understood that this present technology is not limited to particular methods, reagents, compounds, compositions, labeled compounds or biological systems, which can, of course, vary. It is also to be understood that the terminology used herein is for the purpose of describing particular aspects only, and is not intended to be limiting. Thus, it is intended that the specification be considered as exemplary only with the breadth, scope and spirit of the present technology indicated only by the appended claims, definitions therein and any equivalents thereof.

The embodiments, illustratively described herein may suitably be practiced in the absence of any element or elements, limitation or limitations, not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the claimed technology. Additionally, the phrase “consisting essentially of” will be understood to include those elements specifically recited and those additional elements that do not materially affect the basic and novel characteristics of the claimed technology. The phrase “consisting of” excludes any element not specified.

In addition, where features or aspects of the disclosure are described in terms of Markush groups, those skilled in the art will recognize that the disclosure is also thereby described in terms of any individual member or subgroup of members of the Markush group. Each of the narrower species and subgeneric groupings falling within the generic disclosure also form part of the present technology. This includes the generic description of the present technology with a proviso or negative limitation removing any subject matter from the genus, regardless of whether or not the excised material is specifically recited herein.

All publications, patent applications, issued patents, and other documents (for example, journals, articles and/or textbooks) referred to in this specification are herein incorporated by reference as if each individual publication, patent application, issued patent, or other document was specifically and individually indicated to be incorporated by reference in its entirety. Definitions that are contained in text incorporated by reference are excluded to the extent that they contradict definitions in this disclosure.

Other embodiments are set forth in the following claims, along with the full scope of equivalents to which such claims are entitled.

While the technology has been particularly shown and described with reference to a preferred embodiment and various alternate embodiments, it will be understood by persons skilled in the relevant art that various changes in form and details can be made therein without departing from the spirit and scope of the invention.

All references, issued patents and patent applications cited within the body of the instant specification are hereby incorporated by reference in their entirety, for all purposes.

Although the technology has been described with reference to embodiments and examples, it should be understood that numerous and various modifications can be made without departing from the spirit of the invention. Accordingly, the technology is limited only by the following claims.

1. A pharmaceutical composition comprising a compound of Formula I: 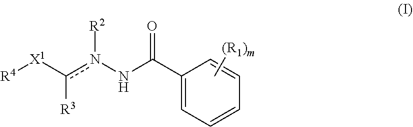
wherein each R¹ and R⁴ is independently selected from the group consisting of halogen, an optionally substituted alkyl, an optionally substituted alkenyl, an optionally substituted alkynyl, an optionally substituted aryl, an optionally substituted heteroaryl, —COOH, alkoxy, —OH, and —NO₂;
R² is hydrogen or absent;
“--” is a pi bond or absent;
R³ is hydrogen, ═O, ═S, an optionally substituted aryl, or an optionally substituted heteroaryl;
m is 0, 1, 2, or 3; and
X¹ is a covalent bond, 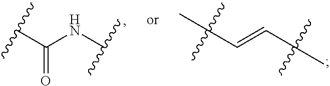
or a stereoisomer or pharmaceutically acceptable salt thereof, and a pharmaceutically acceptable excipient.

2-23. (canceled)

24. A pharmaceutical composition comprising a therapeutically effective amount of a compound selected from: 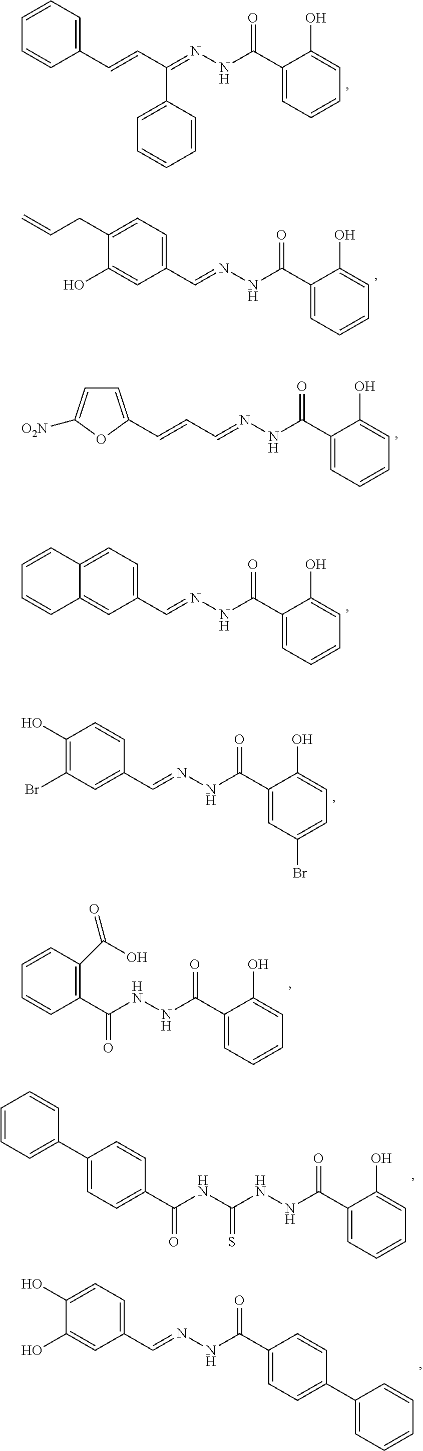 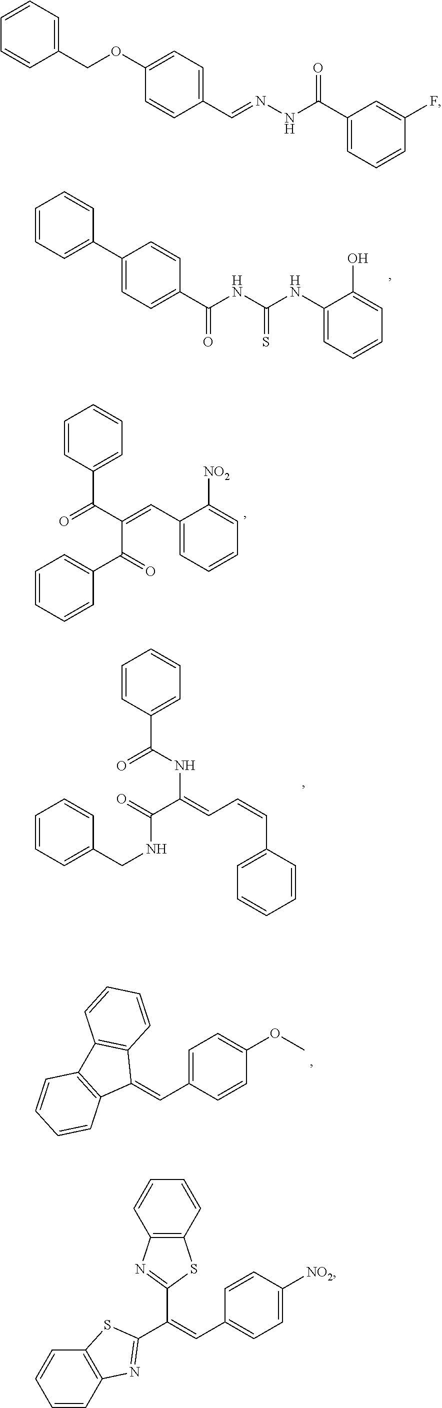 [SMILES: O=C(N/N=C/c1ccc(OCc2ccccc2)cc1)c1cccc(F)c1 ; O=C(NC(=S)Nc1ccccc1O)c1ccc(-c2ccccc2)cc1 ; O=C(C(=Cc1ccccc1[N+](=O)[O-])C(=O)c1ccccc1)c1ccccc1 ; O=C(NCc1ccccc1)/C(=C/C=C\c1ccccc1)NC(=O)c1ccccc1 ; COc1ccc(C=C2c3ccccc3-c3ccccc32)cc1 ; O=[N+]([O-])c1ccc(C=C(c2nc3ccccc3s2)c2nc3ccccc3s2)cc1] 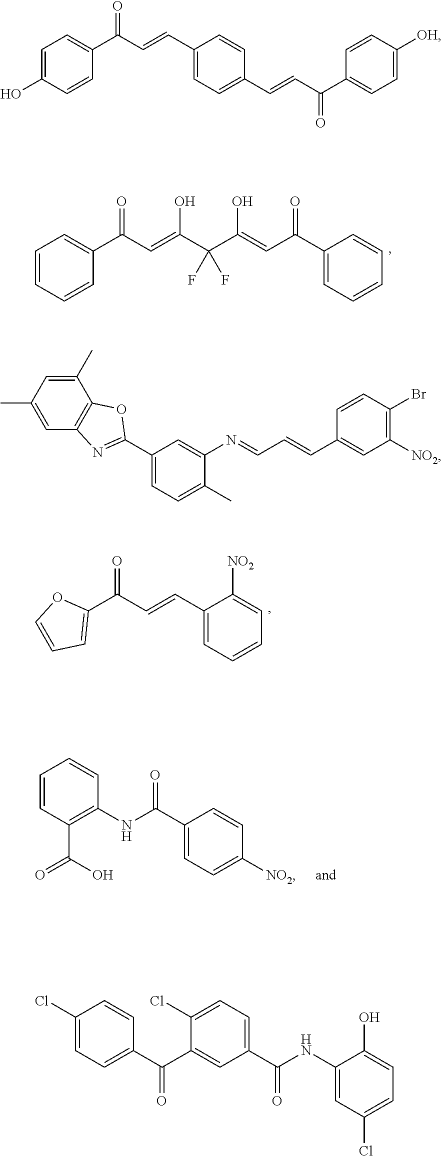 [SMILES: O=C(/C=C/c1ccc(/C=C/C(=O)c2ccc(O)cc2)cc1)c1ccc(O)cc1 ; O=C(/C=C(\O)C(F)(F)/C(O)=C/C(=O)c1ccccc1)c1ccccc1 ; Cc1cc(C)c2oc(-c3ccc(C)c(/N=C/C=C/c4ccc(Br)c([N+](=O)[O-])c4)c3)nc2c1 ; O=C(/C=C/c1ccccc1[N+](=O)[O-])c1ccco1 ; O=C(Nc1ccccc1C(=O)O)c1ccc([N+](=O)[O-])cc1 ; O=C(Nc1cc(Cl)ccc1O)c1ccc(Cl)c(C(=O)c2ccc(Cl)cc2)c1]
or a stereoisomer or pharmaceutically acceptable salt thereof, and a pharmaceutically acceptable excipient.

25. (canceled)

26. The composition of claim 24, wherein the therapeutically effective amount is an amount effective for allosteric inhibition of SOS1.

27. The composition of claim 24, wherein the therapeutically effective amount is an amount effective for allosteric inhibition of SOS1 in an oncogenic cell.

28. The composition of claim 27, wherein the oncogenic cell is present due to a Ras-mediated cancer.

29. The composition of claim 28, wherein the Ras-mediated cancer is pancreatic cancer or acute myeloid leukemia.

30. A method of treating a cancer, comprising administering to a subject in need thereof, the pharmaceutical composition according to claim 1.

31. The method of claim 30, wherein the cancer is an oncogenic Ras-mediated cancer.

32. (canceled)

33. The method of claim 30, wherein the concentration in the subject upon administration is no greater than about 500 μM.

34. (canceled)

35. (canceled)

36. A method of testing a compound for an ability to inhibit a Ras-mediated cancer comprising:
contacting a Ras-mediated cancer cell culture with a test compound;
determining the percent cell survival of the cells within the Ras-mediated cancer cell culture;
identifying a compound as having an ability to inhibit a Ras-mediated cancer when the percent cell survival of the cells within the Ras-mediated cancer cell culture is below a threshold amount.

37. (canceled)

38. (canceled)

39. The method of claim 36, wherein the compound binds to the allosteric site of SOS1.

40. The method of claim 36, wherein the threshold amount for the percent cell survival of the cells within the Ras-mediated cancer cell culture is the percent cell survival of the cells within the Ras-mediated cancer cell culture when contacted with NSC-70220.

41. The method of claim 36, wherein the threshold amount for the percent cell survival of the cells within the Ras-mediated cancer cell culture is 20 percent or less.

42. (canceled)

43. The method of claim 36, further comprising:
contacting a wild-type cell culture with a test compound;
determining the percent cell survival of the cells within the wild-type cell culture;
identifying a compound as specifically binding the allosteric inhibition site of SOS1 when the percent cell survival of the cells within the wild-type cell culture is above a threshold amount.

44. The method of claim 43, wherein the threshold amount for the percent cell survival of the cells within the wild-type cell culture is 50 percent or more.

45. (canceled)

46. The method of claim 43, wherein the wild-type cell culture comprises the same cell type as the Ras-mediated cancer cell culture.

47. The method of claim 43, wherein the wild-type cell culture differs from the Ras-mediated cancer cell culture by virtue of the cells in the wild-type cell culture comprising wild-type Ras and the cells in the Ras-mediated cancer cell culture comprising oncogenic Ras.

48. (canceled)

49. The method of claim 36, wherein the concentration of the compound is about 1 μM to about 500 μM.

50. (canceled)

51. The method of claim 36, wherein the percent cell survival of the cells within the Ras-mediated cancer cell culture is the percent cell survival after 1-7 days.

52. (canceled)

53. The method of claim 43, wherein the percent cell survival of the cells within the wild-type cell culture is the percent cell survival after 1-7 days.

54. (canceled)
